(19) United States
(12) Patent Application Publication
(10) Pub. No.: US 2025/0215149 A1
(43) Pub. Date: Jul. 3, 2025

(54) IMPROVED METHOD FOR THE DEPOLYMERIZATION OF POLYETHYLENE TEREPHTHALATE

(71) Applicant: EVONIK OPERATIONS GMBH, Essen (DE)

(72) Inventors: Philip Heinrich REINSBERG, Rheinbach (DE); Johannes RUWWE, Hanau (DE); Jörn Klaus Erich WOLF, Essen (DE); Felix GÄRTNER, Haltern am See (DE); Jutta MALTER, Darmstadt (DE); Dirk ROETTGER, Köln (DE)

(73) Assignee: EVONIK OPERATIONS GMBH, Essen (DE)

(21) Appl. No.: 18/853,152

(22) PCT Filed: Nov. 18, 2022
(86) PCT No.: PCT/EP2022/082369

§ 371 Date: Oct. 1, 2024

(30) Foreign Application Priority Data
Apr. 4, 2022 (EP) 22166568.0

(51) Int. Cl. C08G 63/78; C07C 67/297

(52) U.S. Cl. CPC C08G 63/78; C07C 67/297

(57) ABSTRACT

The present invention relates to a method for the depolymerization of polyethylene terephthalate (PET), in which method PET is reacted with sodium glycolate or potassium glycolate which has been obtained via reactive distillation, to form a mixture M1 comprising bis(2-hydroxyethyl) terephthalate (BHET). The method according to the invention is characterized in that BHET forms a particularly high proportion of the cleavage products in the mixture M1. As a result, the method according to the invention provides a high yield of BHET, which can be used directly to produce PET again. The present invention therefore also relates to a method for recycling PET, in which method the BHET that is obtained in the method for the depolymerization of PET and, if necessary, has been further purified from M1, is repolymerized to form PET.

The present invention relates to a method of depolymerization of polyethylene terephthalate (=“PET”), in which PET is reacted with sodium ethyleneglycolate or potassium ethyleneglycolate that has been obtained by a reactive distillation to give a mixture M₁ comprising bis-2-hydroxyethyl terephthalate (=“BHET”; CAS No.: 959-26-2).

It is a feature of the method according to the invention that BHET forms a particularly high proportion among the cleavage products in the mixture M₁. As a result, the method according to the invention affords a high yield of BHET, which can be used directly for new production of PET.

The present invention thus also relates to a method of recycling PET, in which the BHET obtained in the method of depolymerization of PET, optionally after further purification from M₁, is polymerized again to give PET.

### BACKGROUND OF THE INVENTION

Polyethylene terephthalate (=“PET”) is one of the most important plastics which is used in textile fibres, as films, and as material for plastic bottles. In 2007 alone, the volume used in plastic bottles was ˜10⁷ t (W. Caseri, Polyethylenterephthalate, RD-16-03258 (2009) in F. Böckler, B. Dill, G. Eisenbrand, F. Faupel, B. Fugmann, T. Gamse, R. Matissek, G. Pohnert, A. Ruhling, S. Schmidt, G. Sprenger, ROMPP [Online], Stuttgart, Georg Thieme Verlag, January 2022).

On account of its persistence and the volumes of refuse originating from PET, it constitutes one of the greatest environmental challenges at present. The solution to this problem lies in the avoidance and in the efficient reutilization of PET.

The prior art proposes multiple methods of cleavage of PET.

GB 784,248 A describes the methanolysis of PET.

Hydrolytic methods for depolymerization of PET are described by JP 2000-309663 A, U.S. Pat. No. 4,355,175 A and T. Yoshioka, N. Okayama, A. Okuwaki, Ind. Eng. Chem. Res. 1998, 37, 336-340.

The reaction of PET with glycol is described in EP 0723951 A1, U.S. Pat. No. 3,222,299 A, WO 2020/002999 A2, by S. R. Shukla, A. M. Harad, Journal of Applied Polymer Science 2005, 97, 513-517 (“Shukla & Harad” hereinafter) and by N. D. Pingale, S. R. Shukla, European Polymer Journal 2008, 44, 4151-4156.

Shukla & Harad state that PET glycolysis gives rise to bis-2-hydroxyethyl terephthalate (=“BHET”).

This cleavage product may simultaneously be used as reactant for production of new PET.

There is accordingly an interest in methods of depolymerization of PET in which a maximum proportion of BHET is obtained among the cleavage products.

The problem addressed by the present invention was that of providing such a method.

### BRIEF DESCRIPTION OF THE INVENTION

A method that solves the problem addressed by the invention has now surprisingly been found.

The present invention relates to a method of depolymerization of polyethylene terephthalate PET, comprising the following steps:
(a) converting MAOR and glycol in a reactive distillation to obtain a solution S_{AP} comprising glycol and M_{A} glycolate, where M_{A} is an alkali metal selected from sodium, potassium, preferably M_{A}=sodium, and where R is an alkyl radical having 1 to 4, especially 1 to 3, preferably 1 or 2, carbon atoms, and most preferably R=methyl,
(b) reacting the solution S_{AP} with PET to give a mixture M₁ comprising bis-2-hydroxyethyl terephthalate (BHET).

Preferably, S_{AP} is obtained in step (a) by reacting a reactant stream S_{AE1} comprising glycol with a reactant stream S_{AE2} comprising MAOR in countercurrent in a reactive rectification column RR_{A} to give a crude product RP_{A} comprising M_{A} glycolate, ROH, glycol, MAOR, wherein S_{AP} is withdrawn as bottom product stream at the lower end of RR_{A}.

Optionally, a vapour stream S_{AB} comprising ROH, with or without glycol, is withdrawn at the upper end of RR_{A}.

In a further aspect, the present invention relates to a method of recycling PET, in which BHET obtained in the method according to the invention for depolymerization is polymerized in a step (g) to give PET.

It has been found that, surprisingly, the reaction of the PET with the S_{AP} obtained from the alkoxide MAOR by reactive distillation affords a higher proportion of BHET than in conventional methods in which the alkaline alkali metal glycolate solution is obtained by mixing the glycol in the corresponding alkali metal hydroxide.

### DETAILED DESCRIPTION OF THE INVENTION

It has now been found that, surprisingly, the glycolysis of PET proceeds particularly efficiently when sodium glycolate or potassium glycolate that has been obtained by reactive distillation is used. In the reactive distillation according to the invention, the glycolate is obtained by reaction of the corresponding alkali metal alkoxide MAOR with glycol. It has now been observed that, in the method according to the invention, by comparison with the prior art methods in which glycolate that has been obtained by dissolution of the alkali metal hydroxides in glycol is used, a higher proportion of BHET is obtained in the cleavage product.

1. Step (a): Reactive Distillation to Obtain the Solution S_{AP} Comprising Glycol and M_{A} Glycolate

According to the invention, the solution S_{AP} comprising glycol and M_{A} glycolate which is used in the method according to the invention is obtained by means of reactive distillation, by conversion of MAOR and glycol.

M_{A} is an alkali metal selected from sodium, potassium. M_{A} is preferably sodium.

R is an alkyl radical having 1 to 4 carbon atoms, preferably an alkyl radical having 1 to 3 carbon atoms, more preferably methyl or ethyl, most preferably methyl.

Alkyl radicals having 1 to 4 carbon atoms are especially selected from methyl, ethyl, n-propyl, iso-propyl, n-butyl, sec-butyl, tert-butyl, preferably selected from methyl, ethyl, n-propyl, iso-propyl, n-butyl, tert-butyl, more preferably selected from methyl, ethyl, iso-propyl, tert-butyl.

Alkyl radicals having 1 to 3 carbon atoms are selected from methyl, ethyl, n-propyl, iso-propyl, preferably selected from methyl, ethyl, iso-propyl.

Reactive distillation for preparation of alkali metal alkoxides is an important industrial process since alkali metal alkoxides are used as strong bases in the synthesis of numerous chemicals, for example in the production of active pharmaceutical or agrochemical ingredients, and as catalysts in transesterification and amidation reactions.

Alkali metal alkoxides (MOR^{#}) are prepared by means of reactive distillation, typically in a countercurrent distillation column, from alkali metal hydroxides (MOH) and alcohols (R^{#}OH), with removal of the water of reaction formed according to the following reaction <1> together with the distillate:

MOH+R^{#} [symbol] MOR^{#}+H₂O.

Such a method principle is described, for example, in U.S. Pat. No. 2,877,274 A, wherein aqueous alkali metal hydroxide solution and gaseous methanol are conducted in countercurrent in a rectification column. This method is described again in basically unchanged form in WO 01/42178 A1.

The most industrially important alkali metal alkoxides are those of sodium and potassium, especially the methoxides and ethoxides. There are many descriptions of the synthesis thereof in the prior art, for example in EP 1 997 794 A1, WO 2021/148174 A1 and WO 2021/148175 A1.

Methods that are similar, but in which an introducing agent, for example benzene, is additionally used, are described in GB 377,631 A and U.S. Pat. No. 1,910,331 A.

Correspondingly, DE 96 89 03 C describes a method of continuous preparation of alkali metal alkoxides in a reaction column, wherein the water-alcohol mixture withdrawn at the top of the column is condensed and then subjected to a phase separation. The aqueous phase is discarded and the alcoholic phase is returned to the top of the column together with the fresh alcohol. EP 0 299 577 A2 describes a similar method, wherein the water in the condensate is separated off with the aid of a membrane.

Rather than the alkali metal hydroxide, it is also possible to use a different alkali metal alkoxide MOR* in a “transalcoholisation reaction” by reactive distillation and to react it with the alcohol R^{#}OH to give the desired alkali metal alkoxide MOR^{#} and the alcohol R*OH. R*OH here is typically an alcohol having a lower boiling point than R^{#}OH. Typically, methanol is used as R*OH, and the methoxide as MOR*.

Such transalcoholisation reactions are described, for example, in DE 2726491 A1, EP 0776995 or WO 2021/122702 A1.

In a preferred embodiment of the method according to the invention, S_{AP} is obtained in step (a) by reacting a reactant stream S_{AE1} comprising glycol with a reactant stream S_{AE2} comprising M_{A}OR in countercurrent in a reactive rectification column RR_{A} to give a crude product RP_{A} comprising M_{A} glycolate, ROH, glycol, MAOR, wherein S_{AP} is withdrawn as bottom product stream at the lower end of RR_{A}.

Even more preferably, a vapour stream S_{AB} comprising ROH, with or without glycol, is withdrawn at the upper end of RR_{A}.

According to the invention, a “reactive rectification column” is defined as a rectification column in which the reaction according to step (a) of the method according to the invention proceeds at least in some parts. It may also be referred to as “reaction column” for short.

In the preferred embodiment of the method according to the invention, a bottom product stream S_{AP} comprising glycol and M_{A} glycolate is withdrawn at the lower end of RR_{A}. A vapour stream S_{A}B comprising ROH, with or without glycol, is withdrawn at the upper end of RR_{A}.

“Glycol” in the context of the invention is understood to mean ethylene-1,2-diol having the chemical formula HO—CH₂—CH₂—OH (CAS No. 107-21-1).

“M_{A} glycolate” in the context of the invention is understood to mean the salt of the glycol with M_{A}. The term “M_{A} glycolate” comprises at least one of MAO-CH₂—CH₂—OH and MAO-CH₂—CH₂-OMA, preferably at least MAO-CH₂—CH₂—OH, most preferably MAO-CH₂—CH₂—OH and MAO-CH₂—CH₂-OMA.

M_{A} is an alkali metal selected from sodium, potassium, and is preferably sodium.

The reactant stream S_{AE1} comprises glycol. In a preferred embodiment, the proportion by mass of glycol in S_{AE1} is ≥95% by weight, still more preferably 99.5% by weight, where S_{AE1} otherwise comprises especially water, diethylene glycol.

The glycol used as reactant stream S_{AE1} in the preferred embodiment of the method according to the invention may also be commercial glycol having a proportion by mass of glycol of more than 99.5% by weight and a proportion by mass of water of up to 0.03% by weight, up to 0.05% by weight, of diethylene glycol.

In one embodiment of the present invention, the reactant stream S_{AE1} is added in vaporous form to the reactive rectification column RR_{A}.

In an alternative, preferred embodiment of the method according to the invention, glycol is initially charged in the bottom of the reactive rectification column RR_{A} prior to step (a), and then heated to boiling in step (a), which produces a constant reactant stream S_{AE1} in the reactive rectification column RR_{A}. If necessary, glycol is then replenished in the bottom of the reactive rectification column RR_{A} during the performance of step (a).

The reactant stream S_{AE2} comprises MAOR. In a preferred embodiment, S_{AE2} comprises not only MAOR but also at least one further compound selected from ROH, glycol. Even more preferably, S_{AE2} comprises not only MAOR but also ROH, in which case S_{AE2} is an alcoholic solution of MAOR in ROH.

When the reactant stream S_{AE2} comprises MAOR and ROH, the proportion by mass of MAOR based on the total weight of the alcoholic solution in ROH that forms S_{AE2} is especially in the range from 5% to 75% by weight, preferably from 10% to 40% by weight, more preferably from 15% to 35% by weight and even more preferably from 21% to 30% by weight.

In the case that R=methyl, the proportion by mass of MAOR, based on the total weight of the alcoholic solution in ROH that forms S_{AE2}, is especially in the range from 25% to 35% by weight, preferably 30% by weight.

In the case that R=ethyl, the proportion by mass of MAOR, based on the total weight of the alcoholic solution in ROH that forms S_{AE2}, is especially in the range from 15% to 25% by weight, preferably 21% by weight.

The reactant stream S_{AE2} preferably comprises little water, i.e. the portion of water in the reactant stream S_{AE2} is preferably <1% by weight, more preferably <0.2% by weight, even more preferably <0.1% by weight, even more preferably <0.01% by weight, based in each case on the total weight of the reactant stream S_{AE2}.

Step (a) of the method according to the invention is preferably performed in a reactive rectification column (or “reaction column”) RR_{A}.

The reaction column RR_{A} preferably contains internals. Suitable internals are, for example, trays, structured packings or unstructured packings. When the reaction column RR_{A} contains trays, suitable trays are bubble-cap trays, valve trays, tunnel-cap trays, Thormann trays, cross-slit bubble-cap trays or sieve trays. When the reaction column RR_{A} contains trays, it is preferable to choose trays where not more than 5% by weight, more preferably less than 1% by weight, of the liquid trickles through the respective trays. The construction measures required to minimize trickle-through of the liquid are familiar to those skilled in the art. In the case of valve trays, particularly tightly closing valve designs are selected for example. Reducing the number of valves also makes it possible to increase the vapour velocity in the tray openings to twice the value typically established. When using sieve trays it is particularly advantageous to reduce the diameter of the tray openings while maintaining or even increasing the number of openings.

When using structured or unstructured packings, structured packings are preferred in terms of uniform distribution of the liquid.

Step (a) of the method according to the invention may be carried out either continuously or batchwise. It is preferably effected continuously.

“Reaction of a reactant stream S_{AE1} comprising glycol with a reactant stream S_{AE2} comprising MAOR in countercurrent in a reactive rectification column RR_{A}” is achieved in one embodiment of the invention, in particular, by virtue of the feed point for at least a portion of the reactant stream S_{AE1} comprising glycol being located in the reaction column RR_{A} below the feed point for the reactant stream S_{AE2} comprising MAOR.

In an alternative preferred embodiment, the “reaction of a reactant stream S_{AE1} comprising glycol with a reactant stream S_{AE2} comprising MAOR in countercurrent in a reactive rectification column RR_{A}” can also be achieved by virtue of the feed point for at least a portion of the reactant stream S_{AE1} comprising glycol being located in the reaction column RR_{A} above the feed point for the reactant stream S_{AE2} comprising MAOR.

In this embodiment, the reaction column RR_{A} preferably comprises at least 2, in particular 15 to 40, theoretical plates between the feed point for the reactant stream S_{AE1} and the feed point for the reactant stream S_{AE2}.

The reaction column RR_{A} may be operated as a pure stripping column. In that case, the reactant stream S_{AE1} comprising glycol is introduced in vaporous form in the lower region of the reaction column RR_{A}.

Optionally, a portion of the reactant stream S_{AE1} comprising glycol is added in vaporous form below the feed point for the reactant stream S_{AE2} comprising MAOR, but nevertheless at the upper end or in the region of the upper end of the reaction column RR_{A}. This makes it possible to reduce the dimensions of the lower region of the reaction column RR_{A}. When a portion of the reactant stream S_{AE1} comprising glycol is added, especially in vaporous form, at the upper end or in the region of the upper end of the reaction column RR_{A}, preferably only a portion of 10% to 70% by weight, preferably of 30% to 50% by weight, (based in each case on the total amount of glycol used) is fed in at the lower end of the reaction column RR_{A}, and the remaining portion is added, in vaporous form in a single stream or divided into a plurality of substreams, preferably 1 to 10 theoretical plates, more preferably 1 to 3 theoretical plates, below the feed point for the reactant stream S_{AE2} comprising MAOR.

In an alternative embodiment of step (a) of the method according to the invention, “reaction of a reactant stream S_{AE1} comprising glycol with a reactant stream S_{AE2} comprising MAOR in countercurrent in a reactive rectification column RR_{A}” is achieved, in particular, by virtue of glycol being present in the bottom of the reactive rectification column RR_{A} and the feed point for the reactant stream S_{AE2} comprising MAOR being located above the bottom. During step (a) of the method according to the invention, glycol is then heated to boiling in the bottom of RR_{A} and a reactant stream S_{AE1} comprising glycol is produced. S_{AE1} and S_{AE2} are then directed in countercurrent to one another.

In the reaction column RR_{A}, the reactant stream S_{AE1} comprising glycol then reacts with the reactant stream S_{AE2} comprising MAOR to give M_{A} glycolate and ROH, with these products being present in a mixture with the glycol and MAOR reactants since the reaction is an equilibrium reaction. Accordingly, step (a) affords a crude product RP_{A} in the reaction column RR_{A} that comprises not only the M_{A} glycolate and ROH products but also glycol and MAOR.

At the lower end of RR_{A}, the bottom product stream S_{AP} comprising glycol and M_{A} glycolate is then obtained and withdrawn.

At the upper end of RR_{A}, preferably at the top of the column of RR_{A}, in a preferred embodiment of the method according to the invention, a stream of ROH that may or may not still contain glycol, referred to above as “vapour stream S_{AB} comprising ROH, with or without glycol” is withdrawn.

If the vapour stream S_{AB} contains not only ROH but also glycol, glycol is obtained, preferably by distillation, for example in a rectification column. In this embodiment, at least a portion of the glycol obtained in the distillation can be fed back to the reaction column RR_{A} as reactant stream S_{AE1}.

In a preferred embodiment, S_{A}B, when it comprises not only ROH but also glycol, is directed into a rectification column RD_{A} and is separated in RD_{A} into at least one vapour stream S_{OA} comprising ROH which is withdrawn at the upper end of RD_{A}, and at least one stream S_{UA} comprising glycol which is withdrawn at the lower end of RD_{A}.

The amount of glycol encompassed by the reactant stream S_{AE1} is preferably chosen such that said glycol simultaneously serves as a solvent for the M_{A} glycolate obtained in the bottom product stream S_{AP}. The amount of glycol in the reactant stream S_{AE1} is preferably chosen such that the desired concentration of the M_{A} glycolate solution which is withdrawn as bottom product stream S_{AP} comprising glycol and M_{A} glycolate is present in the bottom of the reaction column.

In a preferred embodiment of the method according to the invention, and especially in the cases in which S_{AE2} comprises not only MAOR but also ROH, the ratio of the total weight (mass:unit:kg) of glycol used as reactant stream S_{AE1} to the total weight (mass:unit:kg) of MAOR used as reactant stream S_{AE2} is 1:1 to 50:1, more preferably 2:1 to 40:1, even more preferably 3:1 to 30:1, yet more preferably 5:1 to 13.5:1.

The reaction column RR_{A} in the preferred embodiment of the method according to the invention is operated with or without, preferably with, reflux.

What is meant by “with reflux” is that the vapour stream S_{AB} comprising ROH, with or without glycol, that is withdrawn at the upper end of the respective column, especially the reaction column RR_{A}, is not removed completely. The relevant vapour stream S_{AB} is thus fed at least partly, preferably partly, back to the respective column as reflux, especially to the reaction column RR_{A}. In the cases where such a reflux is established, the reflux ratio is preferably 0.01 to 1, more preferably 0.02 to 0.9, yet more preferably 0.03 to 0.34, especially preferably 0.04 to 0.27 and very especially preferably 0.05 to 0.24, most preferably 0.2.

A reflux ratio is understood generally and in the context of this invention to mean the ratio of the proportion of the mass flow rate withdrawn from the column (kg/h) which is removed from the respective column in liquid form or gaseous form to the proportion of this mass flow rate (kg/h) which is returned back to the column in liquid form (reflux).

A reflux can be established by mounting a condenser at the top of the respective column. For this purpose, in particular, a condenser K_{RRA} is mounted on the reaction column RR_{A}. In the condenser K_{RRA}, the vapour stream S_{AB} is at least partly condensed and fed back to the respective column, especially to the reaction column RR_{A}.

In the embodiment in which a reflux is established in the reaction column RR_{A}, the MAOR used as reactant stream S_{AE2} in the preferred embodiment of the method according to the invention may also be at least partly mixed with the reflux stream, and the resulting mixture may thus be supplied to the reaction column RR_{A}.

In a preferred embodiment of the method according to the invention, step (a) is especially conducted under distillative conditions under which glycol is refluxed.

Step (a) is performed especially at a temperature in the range from 80° C. to 197° C., preferably 100° C. to 197° C., more preferably 120° C. to 140° C., and at a pressure of 0.01 bar abs. to 1 bar abs., preferably in the range from 0.05 bar abs. to 1 bar abs., more preferably in the range from 0.05 bar abs. to 0.15 bar abs., more preferably in the range from 0.05 bar abs. to 0.10 bar abs.

In a more preferred embodiment, the reaction column RR_{A} comprises at least one evaporator which is especially selected from intermediate evaporators V_{ZA} and bottom evaporators V_{SA}. The reaction column RR_{A} more preferably comprises at least one bottom evaporator V_{SA}.

According to the invention, “intermediate evaporators” V_{Z} refer to evaporators above the bottom of the respective column, especially above the bottom of the reaction column RR_{A} (in which case they are referred to as “V_{ZA}”) or of the rectification column RD_{A} which is used in the preferred embodiment and is described in detail further down (in which case they are referred to as “V_{ZRD}”).

In the case of RR_{A}, said evaporators especially evaporate crude product RP_{A} which is withdrawn from the column as side stream S_{ZAA}.

According to the invention, “bottom evaporators” Vs refer to evaporators that heat the bottom of the respective column, especially the bottom of the reaction column RR_{A} or the bottom of the rectification column RD_{A} which is used in the preferred embodiment and is described in detail further down (in which case they are referred to as “V_{SRD}” or “V_{SRD}”). In the case of RR_{A}, said evaporators especially evaporate at least a portion of the bottom product stream S_{AP}. In the case of RD_{A}, said evaporators especially evaporate bottom product stream S_{UA} or a portion of S_{UA}, S_{U}A1.

An evaporator is typically arranged outside the respective reaction column or rectification column.

Suitable evaporators employable as intermediate evaporators and bottom evaporators include for example natural circulation evaporators, forced circulation evaporators, forced circulation flash evaporators, kettle evaporators, falling-film evaporators or thin-film evaporators. Heat exchangers for the evaporator typically employed in the case of natural circulation evaporators and forced circulation evaporators are shell and tube or plate apparatuses. When using a shell and tube exchanger the heat carrier may flow through the tubes with the mixture to be evaporated flowing around the tubes or else the heat carrier may flow around the tubes with the mixture to be evaporated flowing through the tubes. In the case of a falling-film evaporator, the mixture to be evaporated is typically introduced as a thin film on the inside of a tube and the tube is heated externally. In contrast to a falling-film evaporator, a thin-film evaporator additionally comprises a rotor with wipers which distributes the liquid to be evaporated on the inner wall of the tube to form a thin film.

In addition to the recited evaporator types it is also possible to employ any desired further evaporator type known to those skilled in the art and suitable for use on a rectification column.

In the preferred embodiment of the method according to the invention, S_{AP} comprising glycol and M_{A} glycolate is withdrawn as bottom product stream at the lower end of the reaction column RR_{A}.

It is preferable that the reaction column RR_{A} comprises at least one bottom evaporator V_{SA} through which some of the bottom product stream S_{AP} is then passed and glycol is partly removed therefrom, which affords a bottom product stream S_{AP*} having an elevated proportion by mass of M_{A} glycolate compared to S_{AP}.

In particular, in the method according to the invention, S_{AP}, or S_{AP*} if at least one bottom evaporator V_{SA} through which at least some of the bottom product stream S_{AP} is passed is used and glycol is removed at least partly therefrom, has a proportion by mass of M_{A} glycolate in glycol in the range from 1% to 50% by weight, preferably 5% to 35% by weight, more preferably 15% to 35% by weight, most preferably 20% to 35% by weight, in each case based on the total mass of S_{AP}.

The proportion by mass of ROH in S_{AP}/S_{AP*} is preferably <1% by weight, preferably <0.8% by weight, more preferably <0.5% by weight, based on the total mass of S_{AP}.

The proportion by mass of water in S_{AP}/S_{AP*} is preferably <1% by weight, preferably <0.8% by weight, more preferably <0.5% by weight, based on the total mass of S_{AP}.

The proportion by mass of reactant MAOR in S_{AP}/S_{AP*} is preferably <1% by weight, preferably <0.8% by weight, more preferably <0.5% by weight, more preferably <0.005% by weight, based on the total mass of S_{AP}.

The proportion by mass of water in S_{AE1} is preferably <1% by weight, preferably <0.8% by weight, more preferably <0.1% by weight, more preferably <0.005% by weight, based on the total mass of S_{AE1}.

The proportion by mass of water in S_{AE2} is preferably <1% by weight, preferably <0.8% by weight, more preferably <0.1% by weight, more preferably <0.005% by weight, based on the total mass of S_{AE2}.

In an even more preferred embodiment of the method according to the invention, a vapour stream S_{AB} comprising ROH, with or without glycol, is withdrawn at the upper end of RR_{A}.

2. Rectification of the Vapour Stream S_{AB} in a Rectification Column R_{DA} (Preferred)

In a further preferred embodiment, the vapour stream S_{A}B, when it comprises ROH and glycol, is directed into a rectification column RD_{A} and is separated in RD_{A} into at least one vapour stream S_{OA} comprising ROH which is withdrawn at the upper end of RD_{A}, and at least one stream S_{UA} comprising glycol which is withdrawn at the lower end of RD_{A}.

“At least one vapour stream S_{OA} comprising ROH which is withdrawn at the upper end of RD_{A}” shall be understood to mean that the vapour obtained at the upper end of RD_{A} may be withdrawn there as one or more vapour streams.

“At least one stream S_{UA} comprising glycol which is withdrawn at the lower end of RD_{A}” shall be understood to mean that glycol obtained at the lower end of RD_{A} may be withdrawn there as one or more streams.

The vapour stream S_{AB} may be directed into the rectification column RD_{A} via one or more feed points. In the embodiments of the present invention in which the vapour stream S_{AB} is directed into the rectification column R_{DA} as two or more separate streams, it is advantageous when the feed points for the individual streams are at substantially the same height on the rectification column RD_{A}.

In a preferred embodiment of the method according to the invention, the vapour stream S_{AB}, when it comprises ROH and glycol, is separated in a rectification column RD_{A} into a vapour stream S_{OA} comprising ROH which is withdrawn at the upper end of RD_{A} and a stream S_{UA} comprising glycol which is withdrawn at the lower end of RD_{A}.

Another term for “upper end of a rectification column” is “head”.

Another term for “lower end of a rectification column” is “bottom” or “foot”.

The rectification column RD_{A} used may be any rectification column known to those skilled in the art.

The rectification column RD_{A} preferably contains internals. Suitable internals are, for example, trays, unstructured packings or structured packings. Trays used are typically bubble-cap trays, sieve trays, valve trays, tunnel-cap trays or slotted trays. Unstructured packings are generally beds of random packing elements. Random packing elements used are typically Raschig rings, Pall rings, Berl saddles or Intalox® saddles. Structured packings are for example sold under the Sulzer Mellapack® trade name. Apart from the internals mentioned, further suitable internals are known to a person skilled in the art and can likewise be used.

Preferred internals have a low specific pressure drop per theoretical plate. Structured packings and random packing elements have, for example, a significantly lower pressure drop per theoretical plate than trays. This has the advantage that the pressure drop in the rectification column RD_{A} remains as low as possible and the mechanical power of the compressor and the temperature of the glycol/ROH mixture to be evaporated therefore remain low.

When the rectification column RD_{A} contains structured packings or unstructured packings, these may be divided or in the form of an uninterrupted packing. Typically, however, at least two packings are provided, one packing above the feed point for the vapour stream S_{AB} and one packing below the feed point for the vapour stream S_{AB}. It is also possible to provide one packing above the feed point for the vapour stream S_{AB} and two or more trays below the feed point for the vapour stream S_{AB}. If an unstructured packing is used, for example a random packing, the random packing elements are typically disposed on a suitable support grid (for example sieve tray or mesh tray).

In this preferred embodiment, the at least one vapour stream S_{OA} comprising ROH is then withdrawn at the upper end of the rectification column RD_{A}. The preferred mass fraction of ROH in this vapour stream S_{OA} is ≥99% by weight, more preferably 99.6% by weight, yet more preferably ≥99.9% by weight, with the remainder being especially glycol.

Withdrawn at the lower end of RD_{A} in this preferred embodiment is at least one stream S_{UA} comprising glycol which may preferably include <1% by weight, more preferably ≤5000 ppm by weight, yet more preferably ≤1000 ppm by weight of ROH, more preferably ≤100 ppm by weight of ROH.

The withdrawal of at least one vapour stream S_{OA} comprising ROH at the top of the rectification column RD_{A} shall in particular be understood in the context of the present invention to mean that the at least one vapour stream S_{OA} is withdrawn above the internals in the rectification column RD_{A} as a top stream or as a side stream.

The withdrawal of the at least one stream S_{UA} comprising glycol at the bottom of the rectification column RD_{A} shall in particular be understood in the context of the present invention to mean that the at least one stream S_{UA} is withdrawn as bottom stream or at the lower tray of the rectification column RD_{A}.

The rectification column RD_{A} is operated with or without, preferably with, reflux.

“With reflux” shall be understood to mean that the vapour stream S_{OA} withdrawn at the upper end of the rectification column RD_{A} is not completely discharged but rather partly condensed and returned to the respective rectification column RD_{A}. In the cases where such a reflux is established, the reflux ratio is preferably 0.01 to 1, more preferably 0.02 to 0.9, yet more preferably 0.03 to 0.34, especially preferably 0.04 to 0.27 and very especially preferably 0.05 to 0.24, most preferably 0.2.

A reflux may be established by mounting a condenser K_{RD} at the top of the rectification column RD_{A}. The respective vapour stream S_{OA} is partly condensed in the condenser K_{RD} and returned to the rectification column RD_{A}.

3. Step (b): Reaction of PET with the Solution S_{AP}

In step (b) of the method according to the invention, the solution S_{AP} obtained in step (a), comprising glycol and M_{A} glycolate, is reacted with PET to give a mixture M₁ comprising BHET.

### 3.1 PET Starting Material

The PET which is used in step (b) of the method according to the invention may be any PET which has to be depolymerized. Typically, such PET occurs as waste, especially in the home, in industry or in agriculture.

In one embodiment of the method according to the invention, the PET to be depolymerized is thus in a mixture with other plastics, especially at least one plastic selected from polyethylene (“PE”), polyvinylchloride (“PVC”). This is typically the case when PET from plastic wastes is to be depolymerized in the method according to the invention. In this embodiment, the PET is at least partly separated from the other plastics, preferably by sorting, before being subjected to step (b) of the method according to the invention.

In one embodiment of the method according to the invention, the PET is subjected to at least one pretreatment step.

Such pretreatment steps are described, for example, in DE 10032899 C2.

According to the invention, the PET is subjected to at least one pretreatment step selected from chemical pretreatment step, comminution step, before being used in step (b).

In the cases in which the PET is in a mixture with other plastics, the PET is preferably subjected to at least one pretreatment step selected from at least partial separation from other plastics, preferably by sorting, chemical pretreatment step, comminution step, before being used in step (b).

In the cases in which the PET is in a mixture with other plastics, the PET is more preferably first separated at least partly from other plastics, then subjected to at least one chemical pretreatment and finally comminuted.

The chemical pretreatment step is especially a wash step. Such a wash step has the advantage that any impurities, especially food residues, residues of cosmetics and/or bodily secretions (e.g. blood, sperm, faeces), are removed prior to the performance of step (b). Such impurities can lower the efficiency of the reaction in step (b) and/or worsen the purity of the BHET thus obtained.

In the chemical pretreatment step, especially the wash step, the waste is especially heated in a wash solution at a temperature of 30° C. to 99° C., preferably 50° C. to 90° C., more preferably 70° C. to 85° C.

Typical wash solutions are familiar to the person skilled in the art and are preferably selected from:
aqueous solution of a surfactant, preferably a nonionic surfactant;
aqueous solution of an alkali metal hydroxide or alkaline earth metal hydroxide, preferably aqueous NaOH.

The treatment time in the chemical pretreatment step, especially the wash step, is especially 1 min to 12 h, preferably 10 min to 6 h, more preferably 30 min to 2 h, even more preferably 45 to 90 min, most preferably 60 min.

After the treatment of the PET by the chemical pretreatment step, especially the wash step, the aqueous solution is separated off, for example by filtration, and the cleaned PET is preferably washed at least once with water in order to remove residues of the wash solution.

The PET waste thus obtained is then dried, especially in a drying cabinet.

The temperature used for drying here is especially in the range of 30 to 120° C., preferably 50° C. to 100° C., more preferably 60° C. to 90° C., most preferably 80° C.

The comminution step has the advantage that the surface area of the PET available for the reaction in step (b) is increased. This increases the reaction rate of the reaction in step (b). The comminution can be effected in apparatuses known to the person skilled in the art, for example a shredder or a cutting mill.

In a further embodiment of the method according to the invention, the PET is decolorized or coloured in a controlled manner before being subjected to step (b). This can be conducted by methods known to the person skilled in the art, for example decolorization with hydrogen peroxide or dyeing with a dye.

### 3.2 Reaction Conditions

The reaction of the PET with a solution S_{AP} comprising glycol and M_{A} glycolate to give a mixture M₁ can then be effected under the conditions that are familiar to the person skilled in the art.

Preferably, the reaction in step (b) is conducted until, i.e. up to a juncture t_{b} at which, at least P=10%, preferably at least P=20%, more preferably at least P=25%, more preferably at least P=30%, more preferably at least P=40%, more preferably at least P=50%, more preferably at least P=60%, more preferably at least P=70%, more preferably at least P=80%, more preferably at least P=90%, more preferably at least P=95%, even more preferably at least P=99%, of the PET used in step (b) has been converted.

This percentage P is calculated by the following formula:

$$P = ( {n}_{TA} + {n}_{MHET} + {n}_{BHET} ) / {n}_{PET} .$$

n_{PET} here is the molar amount of repeat units of the following structure (≡) in the PET used in step (b):

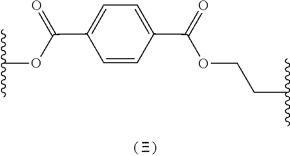

n_{TA} is the molar amount of TA formed in step (b) from commencement of step (b) up to the juncture t_{b}.

n_{MHET} is the molar amount of MHET formed in step (b) from commencement of step (b) up to the juncture t_{b}.

n_{BHET} is the molar amount of BHET formed in step (b) from commencement of step (b) up to the juncture t_{b}.

The structures of compounds BHET, MHET, TA are as follows:

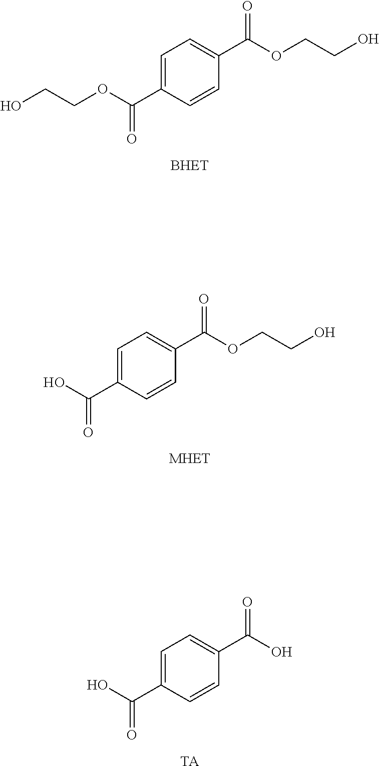

“MHET” also encompasses the corresponding carboxylate of the structure shown.

“TA” also encompasses the corresponding mono- and dicarboxylate of the structure shown.

The reaction in step (b) is especially conducted at a temperature of at least 100° C., preferably at a temperature in the range from ≥100° C. to ≤197° C., more preferably at a temperature in the range from ≥130° C. to ≤197° C., more preferably at a temperature in the range from ≥150° C. to ≤197° C., more preferably at a temperature in the range from ≥175° C. to ≤197° C.

The reaction in step (b) is preferably conducted at the boiling temperature of the glycol. Even more preferably, glycol is refluxed, meaning that glycol is evaporated out of the reaction, condenses and is then returned to the reaction. This refluxing can be established by means familiar to the person skilled in the art, for example in a distillation apparatus.

The total weight of the M_{A} glycolate used in the method based on the total weight of the PET used in the method is especially in the range from 0.1% to 100% by weight, preferably in the range from 0.5% to 80% by weight, more preferably in the range from 1.0% to 50% by weight, more preferably in the range from 1.5% to 25% by weight, more preferably in the range from 2.0% to 10% by weight, more preferably in the range from 2.5% to 6.0% by weight, more preferably 3.5% to 5.0% by weight, most preferably 3.9% by weight.

The reaction in step (b) can be effected with devices familiar to the person skilled in the art.

After step (b) of the method according to the invention has ended, a mixture M₁ is obtained, in which the molar ratio η of the molar amount of BHET (n_{BHET}) to the sum total of the molar amounts of MHET and TA (n_{MHET}+n_{TA}) is in the range of 1:1 to 1000:1, preferably 2:1 to 500:100, more preferably 4:1 to 300:1, even more preferably 10:1 to 100:1, yet more preferably 13:1 to 60:1, yet more preferably 13:1 to 24:1.

$$η = {n}_{BHET} / ( {n}_{MHET} + {n}_{TA} )$$

### 3.3 Preferred Step (c)

In a preferred further step (c), BHET is at least partly separated from M₁. This is even more preferably effected by crystallization and/or distillation. Even more preferably, BHET in step (c) is filtered out of M₁ and then crystallized.

### 4. Process for Recycling of PET

The BHET obtained in the mixture M₁ in the method according to the invention is preferably polymerized to PET in a method of recycling of polyethylene terephthalate in a step (ζ).

This polymerization is known to the person skilled in the art as “polycondensation” and is described, for example, in EP 0 723 951 A1 and by Th. Rieckmann and S. Völker in chapter 2 “Poly(Ethylene Terephthalate) Polymerization—Mechanism, Catalysis, Kinetics, Mass Transfer and Reactor Design” on page 92 of the book “Modern Polyesters: Chemistry and Technology of Polyesters and Copolyesters. Edited by J. Scheirs and T. E. Long, 2003, John Wiley & Sons, Ltd ISBN: 0-471-49856-4”.

In particular, for this purpose, BHET is polymerized back to PET in step (ζ) in the presence of catalysts, which are especially catalysts selected from the group consisting of antimony compounds, preferably Sb₂O₃.

Preferably, the polymerization of BHET to PET in step (ζ) is conducted at least at the boiling temperature of the glycol. In particular, during the polymerization in step (ζ), glycol is removed from the reaction mixture in order to shift the reaction equilibrium to the side of the polymer PET.

More preferably, the polymerization of BHET to PET in step (ζ) is conducted at the boiling temperature of the glycol. Even more preferably, in that case, during the polymerization in step (c), glycol is removed from the reaction mixture in order to shift the reaction equilibrium to the side of the polymer PET.

This is especially achieved by distillation at a pressure of <1 bar, preferably 0.1 mbar, at the simultaneous boiling temperature of the glycol at the respective pressure.

### EXAMPLES

### 1. Inventive Example II

### 1.1 Preparation of the Glycolic Sodium Glycolate Solution by Reactive Distillation

The following apparatus is utilized as distillation apparatus:

The reservoir vessel or bottom used in the distillation apparatus is a heatable 2.5 l jacketed vessel with temperature sensor and vacuum-tight stirrer. Above that is a 25 cm column with Multifill packing and silver mirror (stripping section). Sodium methoxide (“NaOMe”) is metered in above the column by means of a dropping funnel. Above the metering point is a further column that serves to separate off ethylene glycol and methanol vapour (rectifying section). A reflux ratio can be established with the aid of a vapour divider in the upper part of the column, with collection of the distillate in a round-bottomed flask. The round-bottomed flask may be separated from the distillation system via a pressure-equalizing dropping funnel and exchanged. In the rectifying section, a reflux condenser with vacuum connections is attached, by means of which the entire apparatus can be evacuated. The vacuum is generated by means of a rotary vane pump which is connected to the distillation apparatus by two cold traps and a safeguard bottle. The pressure in the distillation apparatus is measured in the safeguard bottle (Buchi vacuum controller), where ventilation is also possible. The bottom reservoir and the column with the Multifill packing are completely surrounded by aluminium foil for insulation in order to assure a uniform temperature in the reactor/column.

The bottom is initially charged with the ethylene glycol and the entire apparatus is evacuated to 50 mbar. Subsequently, the bottoms are heated to boiling temperature, such that a reflux from the rectifying section is established. Subsequently, NaOMe (30% by weight in methanol) is metered in with the aid of a dropping funnel. The metering rate is chosen such that the methanolic NaOMe solution does not reach the bottom (about 2 ml/min).

The methanol added/formed is separated by distillation from ethylene glycol in the rectifying section and collected in the round-bottomed flask. The reflux ratio is 5:1 (5 parts as reflux, 1 part as distillate). The amount distilled off must correspond at least to the amount of methanol added. After the distillative removal, the sodium ethyleneglycolate in the bottoms is subjected to continued distillation for about another 2 hours (the methanol present in the rectifying section is removed at constant vacuum and temperature in order to prevent backflow into the bottom).

After the experiment has ended and been cooled down, the bottom is opened by means of an outlet valve and about 20% by weight solution of sodium glycolate in ethylene glycol is removed.

1.2 PET Depolymerization with Glycolic Sodium Glycolate Solution from the Reactive Distillation

In the method according to the invention, an autoclave is initially charged with 100 g of PET together with 800 g of ethylene glycol. The solution is then heated to 150° C. while stirring. As soon as the temperature of 150° C. has been attained, 19.5 g of 20% sodium glycolate solution in ethylene glycol (corresponding to 0.046 mol) from the reactive distillation is added. The reaction is conducted over the course of five hours, and the reactor output is analysed after cooling. The resultant conversion of BHET (1) and 2-hydroxyethyl terephthalate (=“MHET”) (2) and of terephthalic acid (=“TA”) (3) is determined by gas chromatography.

### 2. Comparative Example V1

In a comparative experiment, an autoclave is initially charged with 100 g of PET together with 800 g of ethylene glycol. The solution is then heated to 150° C. while stirring. The reaction is conducted over the course of five hours, and the reactor output is analysed after cooling. The resultant conversion of BHET (1) and MHET (2) and of TA (3) is determined by gas chromatography.

### 3. Comparative Example V2

In a comparative experiment, an autoclave is initially charged with 100 g of PET together with 800 g of ethylene glycol. The solution is then heated to 150° C. while stirring. As soon as the temperature of 150° C. has been attained, 3.7 g of 50% by weight NaOH solution in water (corresponding to 0.046 mol) is added. The reaction is conducted over the course of five hours, and the reactor output is analysed after cooling. The resultant conversion of BHET (1) and MHET (2) and of TA (3) is determined by gas chromatography.

### 4. Result

Comparison of the content of BHET, MHET and TA in the depolymerized product in Inventive Example E1 and Comparative Examples V1, V2 shows that the depolymerization using the glycolic sodium glycolate solution obtained by reactive distillation affords a higher proportion of BHET. This is advantageous since more product is available as a result, which can be converted directly in a polycondensation to new PET product.

1-15. (canceled)

16. A method for depolymerizing polyethylene terephthalate (PET), comprising the following steps:
(a) converting M_{A}OR and glycol in a reactive distillation to obtain a solution S_{AP} comprising glycol and M_{A} glycolate, where M_{A} is either sodium or potassium, and wherein R is an alkyl radical having 1 to 4 carbon atoms;
(b) reacting the solution S_{AP} with PET to give a mixture M₁ comprising bis-2-hydroxyethyl terephthalate (BHET).

17. The method of claim 16, wherein S_{AP} is obtained in step (a) by reacting a reactant stream S_{AE1} comprising glycol, with a reactant stream S_{AE2} comprising M_{A}OR, in countercurrent in a reactive rectification column RR_{A} to give a crude product RP_{A} comprising M_{A} glycolate, ROH, glycol, and M_{A}OR, wherein S_{AP} is withdrawn as a bottom product stream at the lower end of RR_{A}.

18. The method of claim 17, wherein a vapour stream S_{AB} comprising ROH, with or without glycol, is withdrawn at the upper end of RR_{A}.

19. The method of claim 18, wherein S_{AB} comprises ROH and glycol, and is directed into a rectification column RD_{A} where it is separated into at least one vapour stream S_{OA} comprising ROH which is withdrawn at the upper end of RD_{A}, and at least one stream S_{UA} comprising glycol which is withdrawn at the lower end of RD_{A}.

20. The method of claim 16, wherein the reaction in step (b) is conducted until at least P=10% of the PET used in step (b) has been converted.

21. The method of claim 16, wherein the content of ROH in S_{AP} is <1% by weight.

22. The method of claim 16, wherein the reaction of step (b) is performed at the boiling temperature of the glycol.

23. The method of claim 16, wherein a sufficient amount of S_{AP} is used in step (b) so that the total weight of the M_{A} glycolate used in step (b), based on the total weight of the PET used in step (b), is in the range from 0.1% to 100% by weight.

24. The method of claim 16, wherein BHET is at least partly separated from M₁ in a further step (c).

25. The method of claim 24, wherein the at least partial separation of BHET from M₁ in step (c) is effected by crystallization and/or distillation.

26. The method of claim 16, wherein the PET is subjected to at least one pretreatment step selected from either a chemical pretreatment step, or a comminution step, before being used in step (b).

27. The method of claim 16, wherein the reaction in step (b) is conducted until at leastP=10% of the PET used in step (b) has been converted.

28. The method of claim 19, wherein the content of ROH in S_{AP} is <1% by weight.

29. The method of claim 21, wherein step (b) is performed at the boiling temperature of the glycol.

30. The method of claim 16, wherein a sufficient amount of S_{AP} is used in step (b) so that the total weight of the M_{A} glycolate used in step (b), based on the total weight of the PET used in step (b), is in the range from 0.1% to 100% by weight.

31. A method of recycling polyethylene terephthalate PET, in which BHET is obtained by a method according to claim 16, and the BHET thus obtained is polymerized to PET in a step (ζ).

32. The method of claim 31, wherein the polymerization of BHET to PET in step (ζ) is conducted at least at the boiling temperature of the glycol.

33. The method of claim 31, wherein the polymerization in step (ζ) is performed in the presence of a catalyst.

34. The method of claim 33, wherein the catalyst is an antimony compound.

35. The method of claim 32, wherein the polymerization in step (ζ) is performed in the presence of a catalyst.
